(19) United States
(12) Patent Application Publication
(10) Pub. No.: US 2026/0056217 A1
(43) Pub. Date: Feb. 26, 2026

(54) NORMALIZATION METHODS

(71) Applicant: LIFE TECHNOLOGIES CORPORATION, Carlsbad, CA (US)

(72) Inventors: Alexia Bachir, Eugene, OR (US); Andrew Dix, Eugene, OR (US)

(21) Appl. No.: 19/105,295

(22) PCT Filed: Sep. 21, 2023
(86) PCT No.: PCT/US2023/074789

§ 371 Date: Feb. 20, 2025

Related U.S. Application Data
(60) Provisional application No. 63/377,167, filed on Sep. 26, 2022.

(51) Int. Cl. G01N 33/96

(52) U.S. Cl. CPC G01N 33/96

(57) ABSTRACT

Methods and system are described for normalizing and creating correction factors for measurements in a fluorometer. To ensure that measurements across multiple channels can be properly compared, some kind of calibration must be done. The same calibrant can be run across all the channels and fluorescence measured. The fluorescence of one channel can be chosen as a reference. A correction factor can be calculated for each channel and used for an extended period of time, possibly even the lifetime of the instrument.

### CROSS-REFERENCE TO RELATED APPLICATIONS

This application claims the benefit under 35 U.S.C. § 119(e) of U.S. Provisional Application No. 63/377,167 filed Sep. 26, 2022. The entire contents of the aforementioned application are incorporated by reference herein.

### TECHNICAL FIELD

The present disclosure is directed to normalization methods for fluorometers.

### BACKGROUND OF THE INVENTION

Fluorescence is the emission of light, often in the visible range, by a compound in response to its excitation by higher energy electromagnetic radiation. As excited compounds return to a normal or baseline excitation state, the excess energy is released in the form of light, typically at a less energetic wavelength than that used for excitation. In application, the emission light signals produced during fluorescence can inform the identity and/or concentration of certain compounds within a sample. A fluorometer is one example of an analytical instrument that uses excitation and emission spectra and intensities to analyze biological samples. Using a fluorometer, the presence and concentration of compounds, such as nucleic acids and some proteins, can be determined, whether outright or as part of analysis workflows for DNA, RNA and proteins. Example applications include cloning, sequencing, transfection, qPCR, and protein assays.

In conventional fluorometers, ultraviolet excitation light is produced by an excitation light source (e.g., mercury/xenon lamp, LED, laser) that can provide an intense and consistent source of radiation, thereby allowing saturation of the excitable compounds. The excitation light may be collimated to improve excitation efficiency and then directed toward a biological sample of interest. Fluorescent samples, or fluorescing reagents bound to non-fluorescing samples, become activated through exposure to the excitation light, causing the sample to fluoresce. This fluorescing emission light is received at a photodetector, and these measurements of the amount, intensity, and/or distribution of light can be used to identify and/or approximate concentrations of analyte within the sample.

Certain fluorometers are multi-channel, meaning that multiple samples can be run at a single time. Each channel may have its own light source or have a shared light source that is split into discrete channels, sample holder and other components. With instruments of this nature, each channel becomes its own unique measurement device wherein a single sample would read different when measured on different channels of the same instrument. This lack of cohesion across channels, if not properly addressed, will cause systematic error between measurements. Therefore, to ensure that samples across different channels can be properly compared, it is necessary to calibrate each channel prior to use. This can be time consuming and can use up precious resources. A need, therefore, exists to provide a universal normalization for multi-channel fluorometers.

### BRIEF SUMMARY OF THE INVENTION

One embodiment under the present disclosure comprises a method of normalizing fluorescence measurements for a multi-channel fluorometer. The method comprises receiving a calibrant in a plurality of receptacles of the multi-channel fluorometer; exciting the plurality of receptacles with one or more light sources; and measuring a fluorescence level of each calibrant in the plurality of receptacles. It further comprises using the fluorescence level of the calibrant in a first receptacle of the plurality of receptacles to generate a correction factor for the first receptacle; creating correction factors for each of the other of the plurality of receptacles using the fluorescence level from the first receptacle; storing the correction factors; and applying the correction factors to subsequent measurements of fluorescence levels in each respective receptacle of the plurality of receptacles.

Another embodiment comprises a multi-channel fluorometer. The fluorometer comprises one or more receptacles configured to receive a test solution therein; one or more light sources configured to provide excitation to the one or more receptacles; and one or more photodetectors, each photodetector configured to receive emission light from one of the excited receptacles and to measure a fluorescence level of the emission light. It also comprises a non-transitory computer-readable storage medium coupled to the one or more receptacles, light sources and photodetectors, having stored thereon a computer program which, when executed on at least one processor, causes the at least one processor to carry out a method comprising the steps of: receiving an indication of a calibrant being placed in the one or more receptacles of the multi-channel fluorometer; exciting the calibrant in the one or more receptacles with the one or more light sources; measuring the fluorescence level of each calibrant in the one or more receptacles; using the fluorescence level of the calibrant in a first receptacle of the one or more receptacles to generate a correction factor for the first receptacle; creating correction factors for each of the other of the one or more receptacles using the fluorescence level from the first receptacle; storing the correction factors; and applying the correction factors to subsequent measurements of fluorescence levels in each respective receptacle of the one or more receptacles.

A further embodiment comprises a method of normalizing fluorescence measurements. The method comprises receiving a plurality of fluorescence measurements for a plurality of receptacles in a fluorometer; using the fluorescence measurement of a first receptacle of the plurality of receptacles to generate a correction factor for the first receptacle; and creating correction factors for each of the other of the plurality of receptacles using the fluorescence measurement from the first receptacle. It further comprises storing the additional correction factors; and applying the correction factors to a subsequent plurality of fluorescence measurements in each respective receptacle of the plurality of receptacles.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiments disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawings, in which:

FIG. 1 illustrates a prior art fluorometer;

FIG. 2 illustrates a fluorometer embodiment under the present disclosure;

FIG. 3 illustrates a fluorometer embodiment under the present disclosure;

FIGS. 4A-D illustrate the application of correction factors to fluorescence measurements under the present disclosure;

FIG. 5 illustrates the application of correction factors when using multiple replicates of standards under the present disclosure;

FIG. 6 shows a flow-chart of a method embodiment under the present disclosure;

FIG. 7 shows a flow-chart of a method embodiment under the present disclosure;

FIG. 8 shows a flow-chart of a method embodiment under the present disclosure; and FIG. 9 shows a flow-chart of a method embodiment under the present disclosure.

### DETAILED DESCRIPTION OF THE INVENTION

Before describing various embodiments of the present disclosure in detail, it is to be understood that this disclosure is not limited to the parameters of the particularly exemplified systems, methods, apparatus, products, processes, and/or kits, which may, of course, vary. Thus, while certain embodiments of the present disclosure will be described in detail, with reference to specific configurations, parameters, components, elements, etc., the descriptions are illustrative and are not to be construed as limiting the scope of the claimed invention. In addition, the terminology used herein is for the purpose of describing the embodiments and is not necessarily intended to limit the scope of the claimed invention.

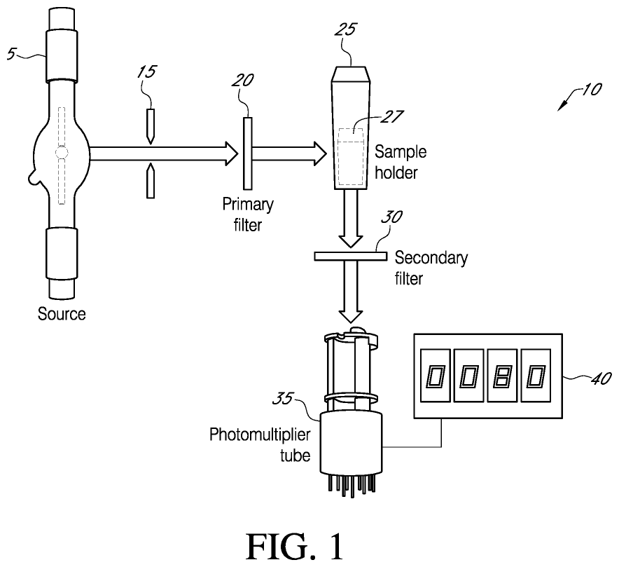

Fluorometers are devices that can measure parameters of visible spectrum fluorescence, such as intensity and wavelength distribution of emission spectrum resulting from excitation by a spectrum of light. This can allow the identification and measurement of specific molecules in a medium. A basic fluorometer 10 is shown in FIG. 1. Fluorometer 10 comprises a light source 5 that directs light through attenuator 15, through a primary filter 20 and onto a sample 25 in a sample holder 27. Light reflected from sample 25 can pass through secondary filter 30 before being received at a detector (or photomultiplier) 35. Detector 35 is coupled to readout 40 or other analysis or display components enabling a user to see which elements or materials are present in the sample 25, possibly via charts or graphs. Some of the light from light source 5 will be absorbed and some will be reflected by sample 25. Different materials absorb and reflect different wavelengths of light. By absorbing the reflected light at detector 35 it can be determined what materials are present in the sample 25.

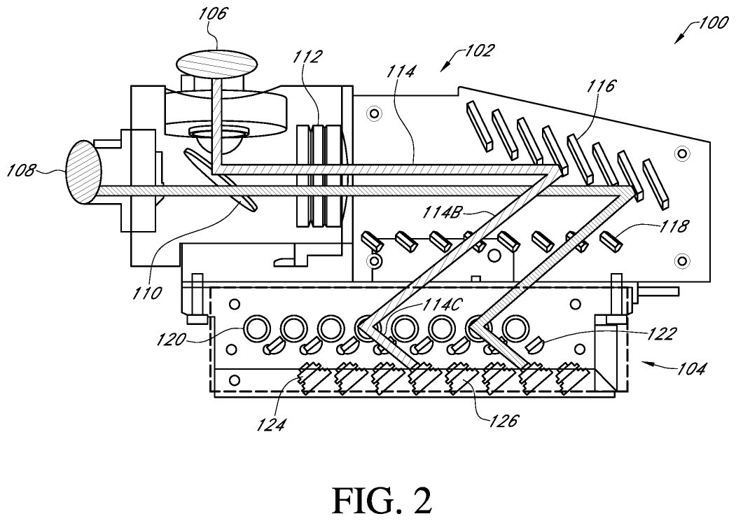

Certain fluorometers can comprise multiple samples and/or multiple sources of light to carry out multiple analyses simultaneously. For example, one embodiment under the present disclosure comprises an 8-channel fluorometer 100, as shown in FIG. 2 and described in detail in U.S. Pat. No. 11,237,108. Fluorometer 100 can measure the fluorescence of up to eight samples simultaneously for the quantification of e.g., DNA, RNA, microRNA, protein, and more.

Fluorometer 100 of FIG. 2 comprises an optical system that includes an excitation module 102 and an emission module 104. The excitation module 102 excites one or more samples (or fluorescent tags within samples) to generate emission light, and the emission module 104 detects the emission light for analysis. The excitation module 102 includes one or more excitation light sources (e.g., LED 106 and/or LED 108), a beam splitter 110 configured to direct one or more beams of excitation light generated by the light source(s) in a first direction (e.g., direction 114) a collimator or attenuator element 112, a plurality of excitation mirrors 116 configured to direct one or more beams of excitation light in a second direction (e.g., direction 114B) toward a plurality of excitation lenses 118 and a plurality of sample receptacles 120 configured to receive sample containers whose excited contents produce emission radiation in a third direction (e.g., direction 114C) toward a plurality of emission lenses 122, a plurality of emission filters 124, and a plurality of photodetectors 126.

The excitation module 102 can utilize a plurality of excitation light sources, such as the blue light emitting diode (LED) 106 and red LED 108 illustrated in FIG. 2, but it will be appreciated that other types or number of excitation light sources, including various excitation wavelengths, may be used. Additional, or alternative excitation light sources include, for example, lasers or mercury/xenon arc lamps. The excitation light source can be selected based on wavelength ranges associated with violet, green, yellow, or orange visible light spectra, and/or non-visible light ranges, such as ultraviolet, near infrared, or infrared lights. In some embodiments, one or more excitation light sources used in the excitation module is selected based on the anticipated identity of analyte to be analyzed within a biological sample.

In some embodiments, the excitation light sources (e.g., LED 106, 108) are specifically tuned to the excitation wavelengths of pre-determined fluorophores. In the illustrated example of FIG. 2, the wavelengths of excitation light produced by the blue LED 106 and the red LED 108, respectively, are selected based on the excitation wavelength of known fluorophores to be used in the analysis of biological samples. Alternatively, a high intensity light source, such as a xenon/mercury arc lamp can be used be used as an excitation light source. Such lamps generate both ultraviolet (UV) light and visible light, making their implementation more practical for non-specific analyses where the exact excitation wavelength or range of wavelengths is unknown. A light source producing a single excitation wavelength or known range of excitation wavelengths can beneficially target a known fluorophore and thereby prevent or reduce inadvertent excitation of non-targeted molecules within the sample. In some embodiments, an excitation filter (e.g., a bandpass filter) can be disposed in front of the excitation light source to narrow the wavelength range of the excitation light, as desired.

As shown in FIG. 2, a beam splitter 110 is used to direct the two beams of excitation light from LEDs 106, 108 along the same light path (e.g., direction 114), thereby reducing the number of components and space used by the excitation module 102. Alternatively, an optical fiber beam combiner, or the like, may be used in place of the beam splitter 110. This illustrated configuration can be beneficial because the excitation module size is reduced, making the overall footprint of the corresponding biological analysis device to also be reduced. Furthermore, the ability to include one or more excitation light sources allows for increased versatility and bespoke configurations of the system for analyzing different samples and/or various types of fluorophores, which may correspond to different ranges of excitation light.

The excitation light, after passing the beam splitter 110, is collimated through a collimator element 112 (e.g., a collimator lens or a concave/parabolic mirror). The collimated beam of excitation light is transmitted along a first direction 114 toward a plurality of excitation mirrors 116. The first direction 114 is generally parallel to the optical axis of the collimator element 112. The excitation light is reflected from the mirrors 116 in the form of a plurality of separate, reflected beams toward a corresponding plurality of excitation lenses 118. Each excitation lens 118 focuses a corresponding reflected beam of excitation light, generating focused beams (e.g., line-focal beams) to illuminate the samples received within the sample receptacles 120 of the emission module 104. The fluorophore(s) within each sample are excited by the focused beams of excitation light and generate emission light.

As shown in FIG. 2, the separate, reflected beams of excitation light are reflected from each corresponding excitation mirror and travel in a second direction (e.g., direction 114B). In some embodiments, the second direction is non-perpendicular relative to the first direction and forms an acute angle with the first direction, thereby causing the reflected beams of excitation light to travel in a direction back toward the collimator element 112. In contrast, some conventional fluorometer optical systems are configured to direct the light path downward, perpendicular to the light path, thus increasing the length of the overall optical system as compared to that provided by the illustrated staggered mirror configuration. Additionally, some conventional fluorometer optical systems do not include reflection excitation mirrors allowing the collimated light to continue on its initial trajectory before passing through any filters and reaching the targeted samples. Such embodiments result in a much larger system than that illustrated in FIG. 2. Therefore, the staggered mirror configuration as shown and described in FIG. 2 beneficially reduces the size of the optical system.

As alluded to above, the plurality of excitation lenses 118 generate focused beams of excitation light that travel from the excitation module 102 to the emission module 104. The emission module 104 includes a series of biological sample receptacles 120 formed into a sample block. As shown, the plurality of sample receptacles 120 are arranged as a series of uniformly spaced receptacles aligned along an axis that is approximately parallel to the first direction 114 of collimated light.

Each receptacle 120 is associated with a respective emission lens 122, emission filter 124, and photodetector 126 (e.g., photodiodes, photomultiplier tubes, CCD/CMOS sensors, etc.). The emission module 104 is configured relative to the excitation module 102 such that each focused beam of excitation light generated by the excitation module 102 travels to, and excites the contents of, a single sample container arranged within a sample receptacle 120 of the emission module 104.

Emission light (e.g., emission radiation, fluorescence radiation) emitted by fluorescing labels or molecules within samples housed in receptacles 120 is collected by individual emission lenses of the plurality of emission lenses 122, ensuring that cross-contamination of emission light from adjacent or multiple samples is prevented or minimized by focusing the emission light along the third direction 114C toward respective photodetectors. The focused emission light then passes through a respective emission filter of the plurality of emission filters 124 to be subsequently detected by respective photodetectors 126. In some embodiments, each photodetector 126 is beneficially disposed at a distance determined by a focal length of the corresponding emission lens 122, so that the emission light beam passing through the emission lens reaches the target photodetector when it is optimally focused to a line-beam. This is beneficial in case one or more of the components are misaligned slightly by ensuring that the emission light reaches at least a portion of the surface of the photodetector lens.

As facilitated by the configuration of the optical components of the excitation module 102 and emission module 104, the emission light is beneficially obtained in a different direction than the excitation light. It is desirable to obtain the emission light in a direction incident to the excitation light so as to avoid receiving direct excitation light at the emission light sensor (e.g., photodetector 126). Emission radiation is emitted in all directions from the excited sample, and most of the excitation light remains directed in the second direction 114B. By placing the emission optics in a direction transverse (e.g., orthogonal) to the second direction 114B, much of the emission light can be observed in the absence of most of the excitation light. Any low-level excitation light reflected in the third direction 114C can be filtered out by emission filters 124 (e.g., bandpass filters) before reaching the photodetector 126.

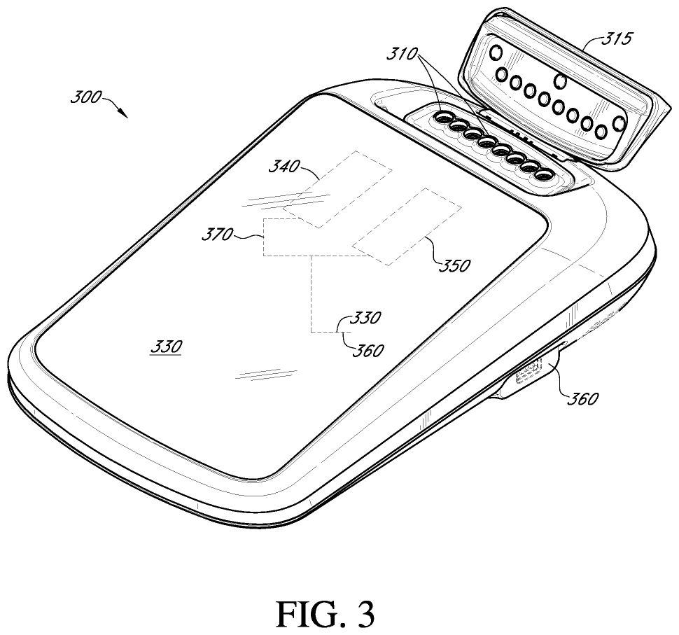

Another view of a possible fluorometer embodiment is shown in FIG. 3. Fluorometer 300 is an 8-channel fluorometer with eight receptacles 310 for samples. Door 315 can hold samples in place and protect the samples during use. Screen 330 can provide a user interface for controlling fluorometer 300 and can comprise a readout for viewing results.

Fluorometer 300 includes processor 340 that can be operatively coupled via a bus 370 to screen 330, a port 360, a memory 350, and/or any other component, or any combination thereof. Certain fluorometers may utilize all or a subset of the components shown in FIG. 3. The level of integration between the components may vary. Further, certain fluorometers may comprise multiple instances of a component, such as multiple processors, memories, transceivers, transmitters, receivers, etc.

Processor 340 is configured to process instructions and data and may be configured to implement any sequential state machine operative to execute instructions stored as machine-readable computer programs in the memory 350. Processor 340 may be implemented as one or more hardware-implemented state machines (e.g., in discrete logic, field-programmable gate arrays (FPGAs), application specific integrated circuits (ASICs), etc.); programmable logic together with appropriate firmware; one or more stored computer programs, general-purpose processors, such as a microprocessor or digital signal processor (DSP), together with appropriate software; or any combination of the above.

Screen 330 may be configured to provide, or may alternatively comprise, an interface or interfaces to an input device, output device, or one or more input and/or output devices. Examples of an output device include a speaker, a sound card, a video card, a display, a monitor, a printer, an actuator, an emitter, a smartcard, another output device, or any combination thereof. Examples of an input device include a touch-sensitive or presence-sensitive display, a camera (e.g., a digital camera, a digital video camera, a web camera, etc.), a microphone, a sensor, a mouse, a trackball, a directional pad, a trackpad, a scroll wheel, a smartcard, and the like. The presence-sensitive display may include a capacitive or resistive touch sensor to sense input from a user. A sensor may be, for instance, an accelerometer, a gyroscope, a tilt sensor, a force sensor, a magnetometer, an optical sensor, a proximity sensor, a biometric sensor, etc., or any combination thereof. An output device may use the same type of interface port as an input device. For example, a Universal Serial Bus (USB) port may be used to provide an input device and an output device.

Port 360 can comprise a power supply connection or a connection to further components. In some embodiments, the power source is structured as a battery or battery pack. Other types of power sources, such as an external power source (e.g., an electricity outlet), photovoltaic device, or power cell, may be used. If port 360 provides coupling to other components, then port 360 can comprise a USB connection, or similar connection. A USB-C connection at port 360, for example, can comprise both power supply coupling and communicative coupling to other components.

Memory 350 can comprise random access memory (RAM), read-only memory (ROM), programmable read-only memory (PROM), erasable programmable read-only memory (EPROM), electrically erasable programmable read-only memory (EEPROM), magnetic disks, optical disks, hard disks, flash drives, or other types of memory. In one example, memory 350 includes one or more application programs, such as an operating system or an application and corresponding data. The memory 350 can be configured to include a number of physical drive units, such as redundant array of independent disks (RAID), flash memory, USB flash drive, external hard disk drive, and others. The memory 350 can allow the fluorometer 300 to access instructions, application programs and the like, stored on transitory or non-transitory memory media, to off-load data, or to upload data. An article of manufacture can be tangibly embodied as on or in the memory 350, which can be or comprise a device-readable storage medium or a non-transitory computer-readable storage medium.

Certain fluorometers, such as in FIG. 2 or FIG. 3, can test multiple samples across multiple different output channels. In a multiple channel fluorometer it can be difficult to ensure accurate results across the multiple channels, multiple samples of the same material, or different material samples. Any difference in the functionality of the multiple channels can distort a comparison of results. Ensuring accurate calibration amongst channels and test samples can be time consuming. For example, if there is a single sample put into multiple tubes, each excited by a different light/laser, then the measured output might not be equal or consistent. A standard curve can be generated using two or more known concentrations of a standard sample. However, when multiple replicates of two, three, four or more concentrations of a standard sample are needed, such as when analyzing endotoxins, many test runs would have to be run to achieve a sufficient amount of data for correction with a standard curve. Embodiments under the present disclosure include methods and systems for normalizing the outputs of multi-channel fluorometers.

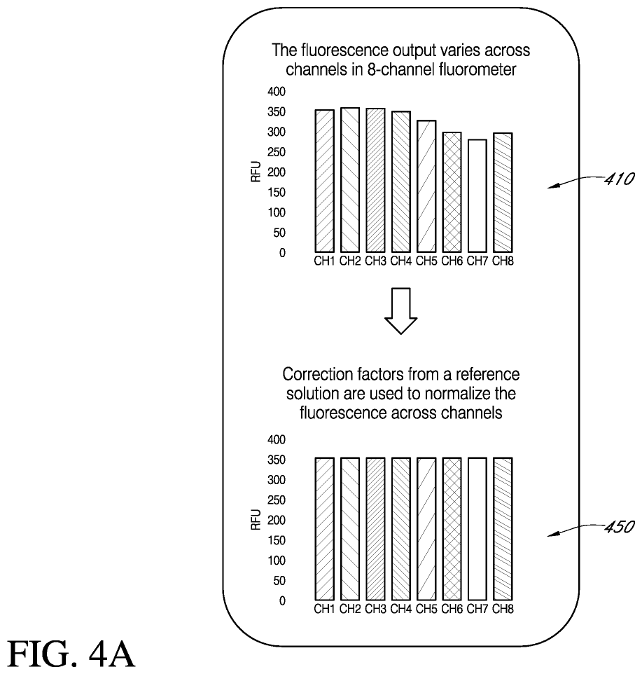

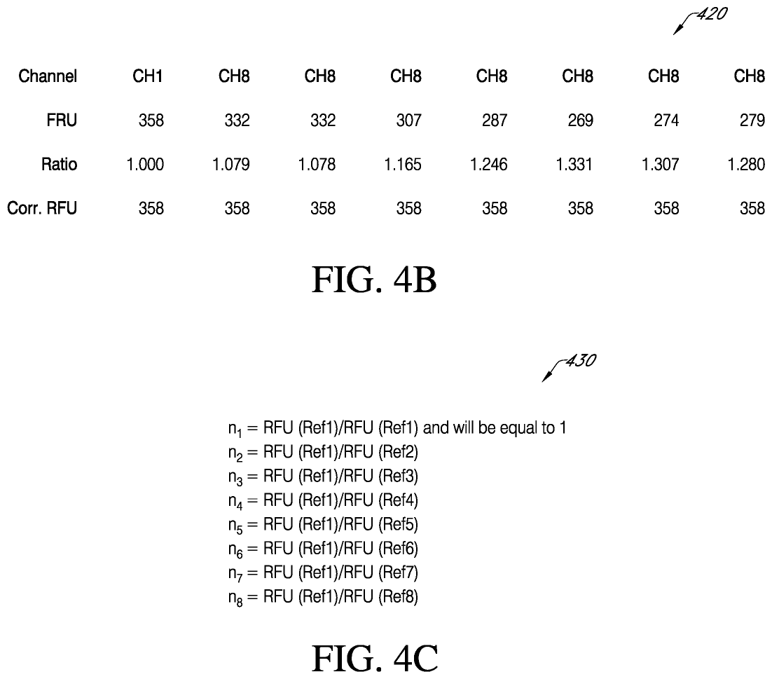

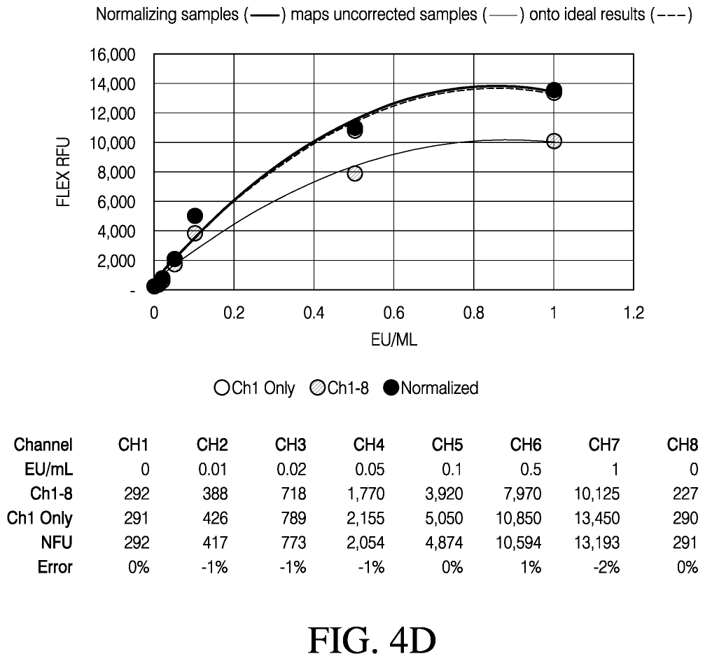

FIG. 4A-C helps to illustrate how a fluorometer can apply correction factors to measured fluorescence values in a multi-channel system. In FIG. 4A, chart 410 shows results of running a set of calibrants, all containing the same material, through an 8-channel fluorometer, such as a QUBIT Flex fluorometer (Thermo Fisher Scientific, Waltham, MA). The calibrant comprises a known concentration of a compound that can be detected by the fluorometer, in this example the calibrant is a 100 nM ALEXA FLUOR 488 dye solution (Thermo Fisher Scientific). Preferably, the compound is a fluorescent dye, fluorophore, quantum dot, fluorescent nanoparticle, or fluorescent gelling polymer. Exemplary fluorophores include, but are not limited to, xanthene, fluorescein, rhodamine, rhodol, roseamine, carbopyranone, indole, indacene, borapolyazaindacene, furan, benzofuran, cyanine, benzocyanine, benzopyrilium, pyrene, coumarin, styryl, squarine, resorufin, anthraquinone, acridine and benzophenoxazine. This is preferably done as a first step in setting up a new fluorometer, after replacing or fixing components, at or after factory calibration, or after a period of use to confirm or adjust correction ratios. In the example shown in FIG. 4A-C, all eight wells of a QUBIT Flex tube strip were filled with 200 μL of the same 100 nM ALEXA FLUOR 488 dye solution and the fluorescence level measurement was obtained for each channel (expressed as RFU). As can be seen in chart 410 and the corresponding table 420 in FIG. 4B, the uncalibrated fluorescence measurement (RFU) for each channel CH1-CH8 is different, though they should be the same because they are measuring the same sample. In FIG. 4C, equations 430 show one embodiment of how to calculate correction factors n₁-n₈ for each of the eight channels. In this embodiment, channel 1 (CH1) is taken to be the reference channel, but another channel could be used. To create each correction factor, the channel 1 fluorescence RFU (Ref1) is divided by the fluorescence of each respective channel RFU (RefX). This will give a correction factor of 1 for the reference channel. In this example, n₁=1. For channel 2, n₂=RFU (Ref1)/RFU (Ref2)=1.079, and so forth. Table 420 shows the resulting correction factor for a given data set. In FIG. 4A, chart 450 shows the results of the fluorescence measurement after normalizing. Because this is a calibrant, each channel should measure the same fluorescence, which is what chart 450 demonstrates. To achieve the numbers in chart 450, the appropriate correction factor is multiplied by the measured RFU of each respective channel. The measured RFU in this example, normalized to channel 1, is 358 RFU (Table 420, “Corr. RFU”). For channel 2 therefore, 358=n₂×RFU (Ref2), or 358=(1.079)×(332). The other channels can be similarly calculated. The correction factors can be stored by the fluorometer and used for subsequent trials.

In another embodiment, the calibration factors are generated by effectively the inverse of the above approach. In this embodiment, channel 1 is again taken to be the reference channel, but another channel could be used. To generate the correction factors, the fluorescence of the calibrant in each respective channel RFU (RefX) is divided by the channel 1 fluorescence RFU (Ref1). The correction factor for each channel can be stored by the fluorometer and used for subsequent trials. To apply the correction factors in a sample assay, the fluorescence of the sample (RFU(Sample)) is divided by the correction factor (CF) for that channel (RefX) as shown in the following equation: RFU(Sample)/CF(RefX). Other embodiments for generating calibration factors can utilize machine learning or can be performed on a secondary software like the cloud. In yet a further embodiment, the correction factors can be applied to a specific value to ensure that all instruments were not only normalized from channel to channel, but from instrument to instrument. This value can be an average of the calibration factors from channels 1-8, or a pre-determined value.

Once the correction factors n₁-n₈ are calculated, they can be used for an extended period of time, possibly even the lifetime of the instrument. If new components are added, or broken ones are fixed, a new calibration, such as in FIG. 4A-C, may need to be run again depending on the type of component. Broken and fixed lenses or light sources, for example, may necessitate repeating the creation of the correction factors. It is possible that, for certain embodiments, it is determined that correction factors can be accurate for a certain number of testing cycles or period of time, e.g., 10,000 hours of use, or 1,000 tests, or 20 months. Once this period of time/use has elapsed, the correction factors may need to be determined again.

To further demonstrate the utility of normalization, a serial dilution series of a 100 nM ALEXA FLUOR 488 dye solution with the concentrations increasing from the blank (i.e., 0 nM) in channel 1 (CH1) to 100 nM in channel (CH7) was prepared with a second blank, (0 nM solution) in channel 8 (CH8), the results of which are shown in the graph and corresponding table of FIG. 4D. The dilution samples were first measured in CH 1 only (open circles, dashed line). This acts as the control as it requires no calibration. Next, the dilution samples were measured using CH1-CH8 without correction factors (hatched circles, thin solid line). As can be seen in FIG. 4D, these values do not align with those obtained from the previous measurement. Next, the fluorescence levels (RFU) from the CH1-CH8 measurements were normalized using correction factors previously determined for this instrument (NFU, filled circles, thick solid line). Advantageously, as can be seen in FIG. 4D, these normalized values overlap well with the measurements obtained from the CH1-only measurements (open circles, dashed line).

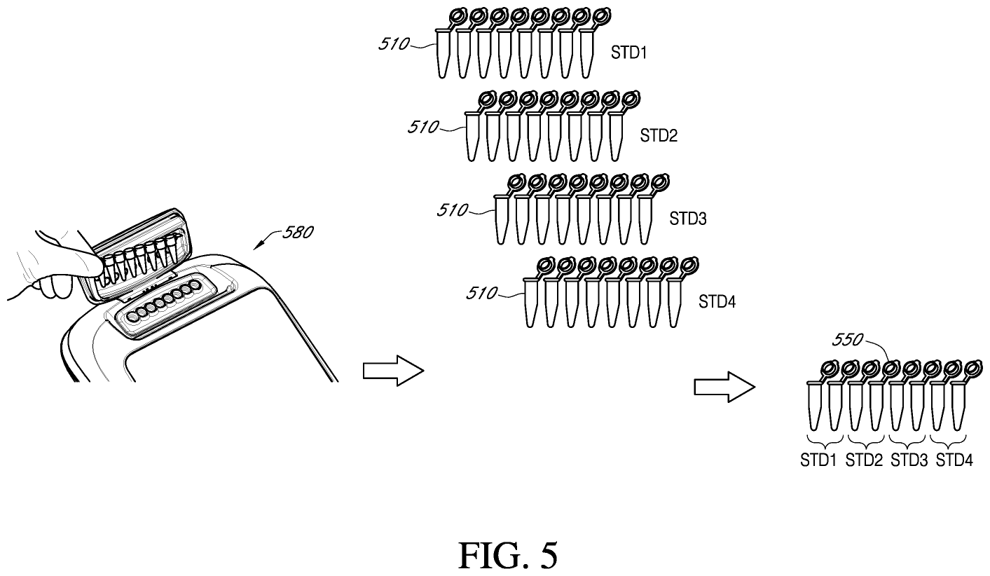

Advantages of the embodiments described herein include cost and time savings. In the prior art, each channel in a multi-channel fluorometer would have to be calibrated before each use. This consumes reagent, samples, time, and energy. Under the present disclosure, a single calibration procedure can provide correction factors that may last for months, years, or the lifetime of the fluorometer. This consumes fewer materials and saves time. The embodiments described can also offer the ability to generate standard curves with multiple replicates (e.g., n≥2) of two or more concentrations of known standards. FIG. 5 illustrates how multiple standards can be run at the same time under embodiments of the present disclosure. Under the prior art, to test multiple standards 510, each one would have to be run separately in multi-channel fluorometer 580. However, under the present disclosure, after determining correction factors as described, multiple standards (STD1-STD4) can be run in a single test strip 550. Test strip 550 can comprise multiple receptacles for each of STD1-STD4. Because the correction factors are already known, the receptacles of the same standard can be compared to each other accurately.

For the example illustrated in FIG. 5, without properly calibrating a multi-channel instrument, such as an 8-channel fluorometer, running four unique concentrations as standards (STD1-STD4) requires four sets of eight fluorescence measurements where each 8-well strip is a unique concentration. For an 8-channel instrument, this is 32 samples. Advantageously, by normalizing the channels using correction factors as provided herein, running four unique concentrations as standards can be done with as few as one fluorescence measurement per concentration. For an 8-channel instrument, this is now only 8 samples. Assays in which this is important are, for example, regulated assays, such as endotoxin, where multiple replicates are required. However, this also proves useful for any other assay as this will reduce the amount of sample or reagent used and time spent using a workflow where the fluorescence output of each channel is separate and not normalized. For example, standard curves using multiple concentrations of known standards can be run simultaneously. In one example, channels 1-8 of an 8-channel fluorometer can be used to run a single replicate of Standards 1-8, where Standards 1-8 are increasing known concentrations of the standard. In another example, channels 1-2 can be used to run two replicates of Standard 1, channels 3-4 to run two replicates of Standard 2, channels 5-6 to run two replicates of Standard 3, and channels 7-8 to run two replicates of Standard 4. In yet another example using an 8-channel fluorometer, four replicates of two concentrations of standards are used where Standard 1 is measured in Channels 1-4 and Standard 2 is measured in Channels 5-8. A further advantage of the correction factors described herein is that a two-strip option (e.g., two QUBIT Flex tube strips, each containing 8 wells) allows for accommodating replicas of three or more (e.g., N=3 or more). For example, four concentrations of standards with four replicates each could be configured as follows: channels 1-4 of strip 1 can be used for Standard 1, channels 5-8 of strip 1 can be used for Standard 2, channels 1-4 of strip 2 can be used for Standard 3, and channels 5-8 of strip 2 can be used for Standard 4. In another example using two strips, two replicates of eight standards can be configured as follows: channels 1-2 of strip 1 can be used for Standard 1, channels 3-4 of strip 1 can be used for Standard 2, channels 5-6 of strip 1 can be used for Standard 3, channels 7-8 of strip 1 can be used for Standard 4, channels 1-2 of strip 2 can be used for Standard 5, channels 3-4 of strip 2 can be used for Standard 6, channels 5-6 of strip 2 can be used for Standard 7, and channels 7-8 of strip 2 can be used for Standard 8.

Reference has been made to an 8-channel fluorometer. However, embodiments under the present disclosure are not limited to 8-channel embodiments. Fluorometer embodiments can include any multi-channel embodiment, such as a 2-channel, 4-channel, 16-channel, 32-channel, 64-channel or other multi-channel device.

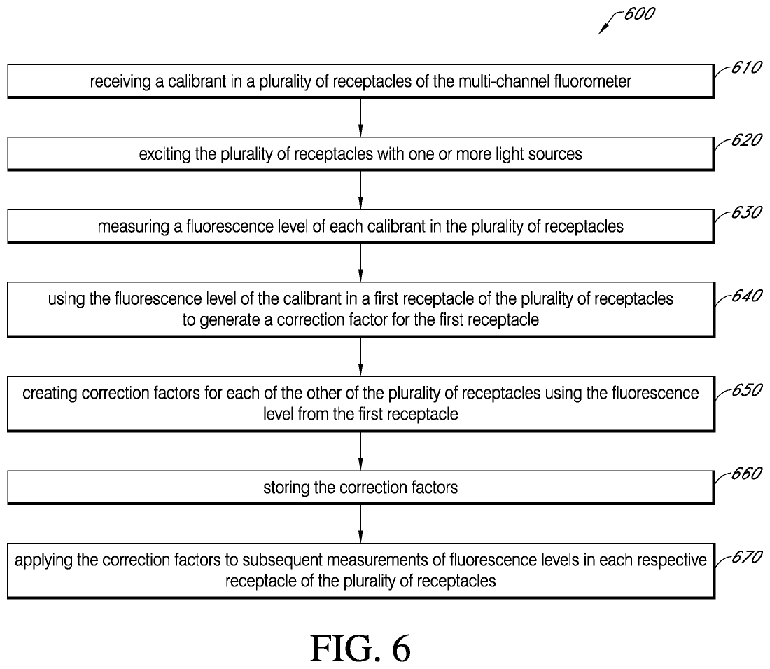

FIG. 6 shows a flow-chart of a possible method embodiment under the present disclosure. Method 600 is a method of normalizing fluorescence measurements for a multi-channel fluorometer. Step 610 is receiving a calibrant in a plurality of receptacles of the multi-channel fluorometer. Step 620 is exciting the plurality of receptacles with one or more light sources. Step 630 is measuring a fluorescence level of each calibrant in the plurality of receptacles. Step 640 is using the fluorescence level of the calibrant in a first receptacle of the plurality of receptacles to generate a correction factor for the first receptacle. Step 650 is creating correction factors for each of the other of the plurality of receptacles using the fluorescence level from the first receptacle. Step 660 is storing the correction factors. Step 670 is applying the correction factors to subsequent measurements of fluorescence levels in each respective receptacle of the plurality of receptacle.

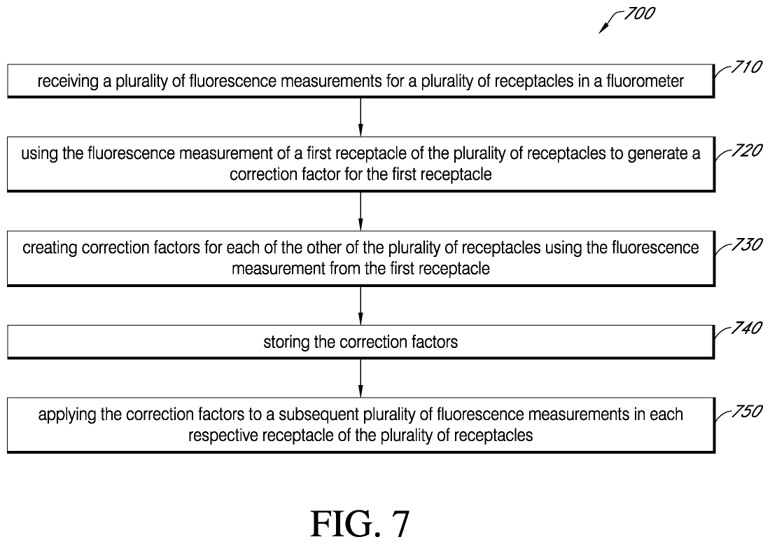

FIG. 7 shows a flow-chart illustrating another possible method embodiment under the present disclosure. Method 700 is a method of normalizing fluorescence measurements. Step 710 is receiving a plurality of fluorescence measurements for a plurality of receptacles in a fluorometer. Step 720 is using the fluorescence measurement of a first receptacle of the plurality of receptacles to generate a correction factor for the first receptacle. Step 730 is creating correction factors for each of the other of the plurality of receptacles using the fluorescence measurement from the first receptacle. Step 740 is storing the correction factors. Step 750 is applying the correction factors to a subsequent plurality of fluorescence measurements in each respective receptacle of the plurality of receptacles.

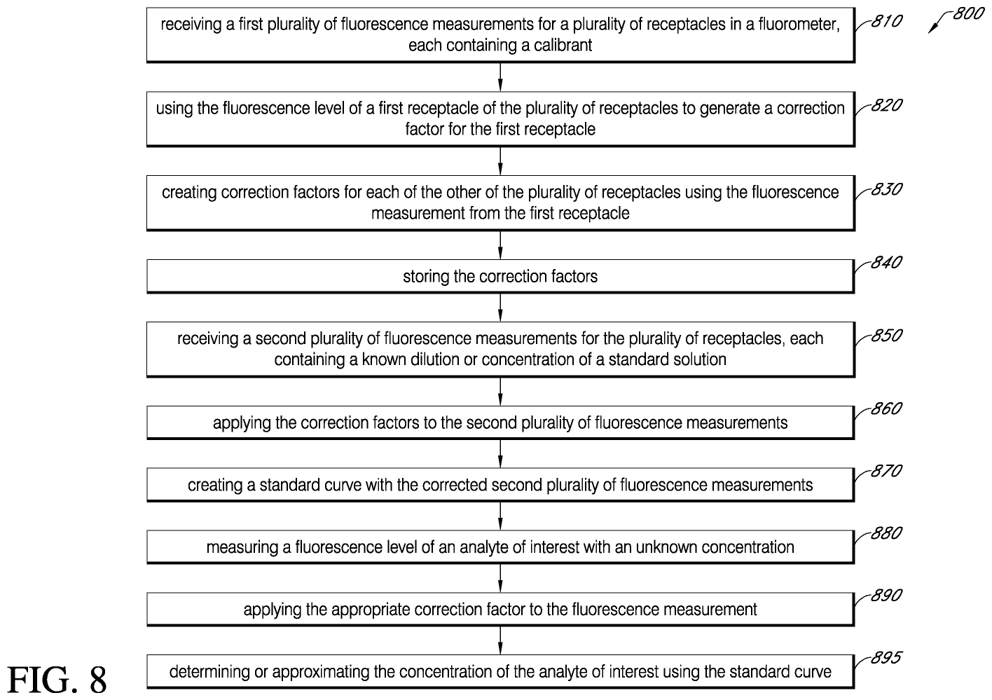

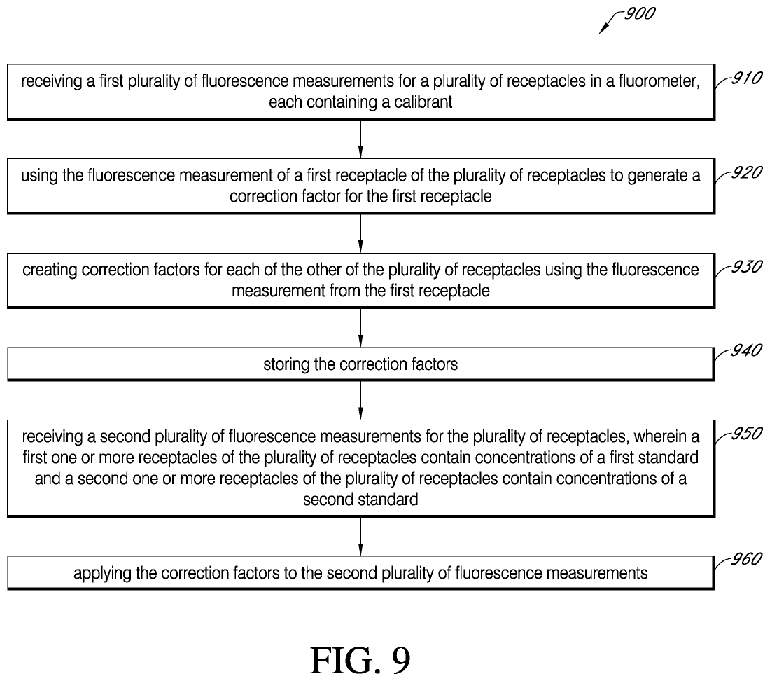

FIG. 8 shows a method of using the normalization correction factors and an array of known concentrations of a standard solution to create a standard curve that can be used to determine the concentration of an analyte of interest. Method 800 includes step 810, which is receiving a first plurality of fluorescence measurements for a plurality of receptacles in a fluorometer, each containing a calibrant. Step 820 is using the fluorescence measurement of a first receptacle of the plurality of receptacles to create a correction factor for the first receptacle. Step 830 is creating correction factors for each of the other receptacles using the fluorescence measurement from the first receptacle. Step 840 is storing the correction factors. Step 850 of method 800 is receiving a second plurality of fluorescence measurements for the plurality of receptacles, each containing a known dilution or concentration of a standard solution. Step 860 is applying the correction factors to the second plurality of fluorescence measurements of those known standard solutions. Step 870 is plotting a standard curve using the normalized data points of the standard solutions to create a curve of concentration to fluorescence. Step 880 is then measuring the fluorescence of a sample containing an analyte of interest. Step 890 is then normalizing the measurement using the correction factor and step 895 plotting the measurement against the standard curve to determine the concentration of the analyte of interest. The analyte of interest can be a biomolecule. In certain embodiments, the biomolecule is chosen from a protein, a peptide, an amino acid, an enzyme, a toxin, a lectin, a lipopolysaccharide, a nanoparticle, a virus, an extracellular vesicle, a nucleic acid, a polynucleotide, an oligonucleotide, a single-stranded DNA, a double-stranded DNA, an RNA or a microRNA.

As stated above, the creation of the standard curve is preferably done using an array of known concentrations of a standard solution. The array can consist of a single instance of each concentration of the standard solution or two or more replicates of each concentration. In certain embodiments, these standard arrays are prepackaged and placed in connected fluorometer tube strips with a visible orientation to ensure that they are properly placed in the device. This type of standard array eliminates the need to transfer standards from individual vials of solution and prevents misplacement of standards into the testing device.

FIG. 9 illustrates another method embodiment 900 under the present disclosure. Method 900 comprises a method of testing fluorescence according to a plurality of standards in a single test. Step 910 is receiving a first plurality of fluorescence measurements for a plurality of receptacles in a fluorometer, each containing a calibrant. Step 920 is using the fluorescence measurement of a first receptacle of the plurality of receptacles to generate a correction factor for the first receptacle. Step 930 is creating correction factors for each of the other of the plurality of receptacles using the fluorescence measurement from the first receptacle. Step 940 is storing the correction factors. Step 950 is receiving a second plurality of fluorescence measurements for the plurality of receptacles, wherein a first one or more receptacles of the plurality of receptacles contain concentrations of a first standard and a second one or more receptacles of the plurality of receptacles contain concentrations of a second standard. Step 960 is applying the correction factors to the second plurality of fluorescence measurements. In one possible embodiment of method 900, there are eight receptacles. And in one possible variation, two receptacles are used for each of four different standards. Other variations are possible.

### Computer Systems of the Present Disclosure

It will be appreciated that computer systems are increasingly taking a wide variety of forms. In this description and in the claims, the terms “controller,” “computer system,” or “computing system” are defined broadly as including any device or system, or combination thereof, that includes at least one physical and tangible processor and a physical and tangible memory capable of having thereon computer-executable instructions that may be executed by a processor. By way of example, not limitation, the term “computer system” or “computing system,” as used herein is intended to include personal computers, desktop computers, laptop computers, tablets, hand-held devices (e.g., mobile telephones, PDAs, pagers), microprocessor-based or programmable consumer electronics, minicomputers, mainframe computers, multi-processor systems, network PCs, distributed computing systems, datacenters, message processors, routers, switches, and even devices that conventionally have not been considered a computing system, such as wearables (e.g., glasses) or cloud-based applications.

The memory may take any form and may depend on the nature and form of the computing system. The memory can be physical system memory, which includes volatile memory, non-volatile memory, or some combination of the two. The term “memory” may also be used herein to refer to non-volatile mass storage such as physical storage media.

The computing system also has thereon multiple structures often referred to as an “executable component.” For instance, the memory of a computing system can include an executable component. The term “executable component” is the name for a structure that is well understood to one of ordinary skill in the art in the field of computing as being a structure that can be software, hardware, or a combination thereof.

For instance, when implemented in software, one of ordinary skill in the art would understand that the structure of an executable component may include software objects, routines, methods, and so forth, that may be executed by one or more processors on the computing system, whether such an executable component exists in the heap of a computing system, or whether the executable component exists on computer-readable storage media. The structure of the executable component exists on a computer-readable medium in such a form that it is operable, when executed by one or more processors of the computing system, to cause the computing system to perform one or more functions, such as the functions and methods described herein. Such a structure may be computer-readable directly by a processor—as is the case if the executable component were binary. Alternatively, the structure may be structured to be interpretable and/or compiled, whether in a single stage or in multiple stages, so as to generate such binary that is directly interpretable by a processor.

The term “executable component” is also well understood by one of ordinary skill as including structures that are implemented exclusively or near-exclusively in hardware logic components, such as within a field programmable gate array (FPGA), an application specific integrated circuit (ASIC), Program-specific Standard Products (ASSPs), System-on-a-chip systems (SOCs), Complex Programmable Logic Devices (CPLDs), or any other specialized circuit. Accordingly, the term “executable component” is a term for a structure that is well understood by those of ordinary skill in the art of computing, whether implemented in software, hardware, or a combination thereof.

The terms “component,” “service,” “engine,” “module,” “control,” “generator,” or the like may also be used in this description. As used in this description and in this case, these terms, whether expressed with or without a modifying clause, are also intended to be synonymous with the term “executable component” and thus also have a structure that is well understood by those of ordinary skill in the art of computing.

While not all computing systems require a user interface, in some embodiments a computing system includes a user interface for use in communicating information from/to a user. The user interface may include output mechanisms as well as input mechanisms. The principles described herein are not limited to the precise output mechanisms or input mechanisms as such will depend on the nature of the device. However, output mechanisms might include, for instance, speakers, displays, tactile output, projections, holograms, and so forth. Examples of input mechanisms might include, for instance, microphones, touchscreens, projections, holograms, cameras, keyboards, stylus, mouse, or other pointer input, sensors of any type, and so forth.

Accordingly, embodiments described herein may comprise or utilize a special purpose or general-purpose computing system. Embodiments described herein also include physical and other computer-readable media for carrying or storing computer-executable instructions and/or data structures. Such computer-readable media can be any available media that can be accessed by a general purpose or special purpose computing system. Computer-readable media that store computer-executable instructions are physical storage media. Computer-readable media that carry computer-executable instructions are transmission media. Thus, by way of example, not limitation, embodiments disclosed or envisioned herein can comprise at least two distinctly different kinds of computer-readable media: storage media and transmission media.

Computer-readable storage media include RAM, ROM, EEPROM, solid state drives (“SSDs”), flash memory, phase-change memory (“PCM”), CD-ROM or other optical disk storage, magnetic disk storage or other magnetic storage devices, or any other physical and tangible storage medium that can be used to store desired program code in the form of computer-executable instructions or data structures and that can be accessed and executed by a general purpose or special purpose computing system to implement the disclosed functionality of the invention. For example, computer-executable instructions may be embodied on one or more computer-readable storage media to form a computer program product.

Transmission media can include a network and/or data links that can be used to carry desired program code in the form of computer-executable instructions or data structures and that can be accessed and executed by a general purpose or special purpose computing system. Combinations of the above should also be included within the scope of computer-readable media.

Further, upon reaching various computing system components, program code in the form of computer-executable instructions or data structures can be transferred automatically from transmission media to storage media (or vice versa). For example, computer-executable instructions or data structures received over a network or data link can be buffered in RAM within a network interface module (e.g., a “NIC”) and then eventually transferred to computing system RAM and/or to less volatile storage media at a computing system. Thus, it should be understood that storage media can be included in computing system components that also, or even primarily, utilize transmission media.

Those skilled in the art will further appreciate that a computing system may also contain communication channels that allow the computing system to communicate with other computing systems over, for example, a network. Accordingly, the methods described herein may be practiced in network computing environments with many types of computing systems and computing system configurations. The disclosed methods may also be practiced in distributed system environments where local and/or remote computing systems, which are linked through a network (either by hardwired data links, wireless data links, or by a combination of hardwired and wireless data links), both perform tasks. In a distributed system environment, the processing, memory, and/or storage capability may be distributed as well.

Those skilled in the art will also appreciate that the disclosed methods may be practiced in a cloud computing environment. Cloud computing environments may be distributed, although this is not required. When distributed, cloud computing environments may be distributed internationally within an organization and/or have components possessed across multiple organizations. In this description and the following claims, “cloud computing” is defined as a model for enabling on-demand network access to a shared pool of configurable computing resources (e.g., networks, servers, storage, applications, and services). The definition of “cloud computing” is not limited to any of the other numerous advantages that can be obtained from such a model when properly deployed.

A cloud-computing model can be composed of various characteristics, such as on-demand self-service, broad network access, resource pooling, rapid elasticity, measured service, and so forth. A cloud-computing model may also come in the form of various service models such as, for example, Software as a Service (“SaaS”), Platform as a Service (“PaaS”), and Infrastructure as a Service (“IaaS”). The cloud-computing model may also be deployed using different deployment models such as private cloud, community cloud, public cloud, hybrid cloud, and so forth.

### Abbreviated List of Defined Terms

To assist in understanding the scope and content of this written description and the appended claims, a select few terms are defined directly below. Unless defined otherwise, all technical and scientific terms used herein have the same meaning as commonly understood by one of ordinary skill in the art to which the present disclosure pertains.

The terms “approximately,” “about,” and “substantially,” as used herein, represent an amount or condition close to the specific stated amount or condition that still performs a desired function or achieves a desired result. For example, the terms “approximately,” “about,” and “substantially” may refer to an amount or condition that deviates by less than 10%, or by less than 5%, or by less than 1%, or by less than 0.1%, or by less than 0.01% from a specifically stated amount or condition.

Various aspects of the present disclosure, including devices, systems, and methods may be illustrated with reference to one or more embodiments or implementations, which are exemplary in nature. As used herein, the term “exemplary” means “serving as an example, instance, or illustration,” and should not necessarily be construed as preferred or advantageous over other embodiments disclosed herein. In addition, reference to an “implementation” of the present disclosure or invention includes a specific reference to one or more embodiments thereof, and vice versa, and is intended to provide illustrative examples without limiting the scope of the invention, which is indicated by the appended claims rather than by the following description.

As used in the specification, a word appearing in the singular encompasses its plural counterpart, and a word appearing in the plural encompasses its singular counterpart, unless implicitly or explicitly understood or stated otherwise. Thus, it will be noted that, as used in this specification and the appended claims, the singular forms “a,” “an” and “the” include plural referents unless the context clearly dictates otherwise. For example, reference to a singular referent (e.g., “a widget”) includes one, two, or more referents unless implicitly or explicitly understood or stated otherwise. Similarly, reference to a plurality of referents should be interpreted as comprising a single referent and/or a plurality of referents unless the content and/or context clearly dictate otherwise. For example, reference to referents in the plural form (e.g., “widgets”) does not necessarily require a plurality of such referents. Instead, it will be appreciated that independent of the inferred number of referents, one or more referents are contemplated herein unless stated otherwise.

As used herein, directional terms, such as “top,” “bottom,” “left,” “right,” “up,” “down,” “upper,” “lower,” “proximal,” “distal,” “adjacent,” and the like are used herein solely to indicate relative directions and are not otherwise intended to limit the scope of the disclosure and/or claimed invention.

### CONCLUSION

It is understood that for any given component or embodiment described herein, any of the possible candidates or alternatives listed for that component may generally be used individually or in combination with one another, unless implicitly or explicitly understood or stated otherwise. Additionally, it will be understood that any list of such candidates or alternatives is merely illustrative, not limiting, unless implicitly or explicitly understood or stated otherwise.

In addition, unless otherwise indicated, numbers expressing quantities, constituents, distances, or other measurements used in the specification and claims are to be understood as being modified by the term “about,” as that term is defined herein. Accordingly, unless indicated to the contrary, the numerical parameters set forth in the specification and attached claims are approximations that may vary depending upon the desired properties sought to be obtained by the subject matter presented herein. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the subject matter presented herein are approximations, the numerical values set forth in the specific examples are reported as precisely as possible. Any numerical values, however, inherently contain certain errors necessarily resulting from the standard deviation found in their respective testing measurements.

Any headings and subheadings used herein are for organizational purposes only and are not meant to be used to limit the scope of the description or the claims.

The terms and expressions which have been employed herein are used as terms of description and not of limitation, and there is no intention in the use of such terms and expressions of excluding any equivalents of the features shown and described or portions thereof, but it is recognized that various modifications are possible within the scope of the invention itemed. Thus, it should be understood that although the present invention has been specifically disclosed in part by preferred embodiments, exemplary embodiments, and optional features, modification and variation of the concepts herein disclosed may be resorted to by those skilled in the art, and such modifications and variations are considered to be within the scope of this invention as defined by the appended items. The specific embodiments provided herein are examples of useful embodiments of the present invention and various alterations and/or modifications of the inventive features illustrated herein, and additional applications of the principles illustrated herein that would occur to one skilled in the relevant art and having possession of this disclosure, can be made to the illustrated embodiments without departing from the spirit and scope of the invention as defined by the items and are to be considered within the scope of this disclosure.

It will also be appreciated that systems, devices, products, kits, methods, and/or processes, according to certain embodiments of the present disclosure may include, incorporate, or otherwise comprise properties or features (e.g., components, members, elements, parts, and/or portions) described in other embodiments disclosed and/or described herein. Accordingly, the various features of certain embodiments can be compatible with, combined with, included in, and/or incorporated into other embodiments of the present disclosure. Thus, disclosure of certain features relative to a specific embodiment of the present disclosure should not be construed as limiting application or inclusion of said features to the specific embodiment. Rather, it will be appreciated that other embodiments can also include said features, members, elements, parts, and/or portions without necessarily departing from the scope of the present disclosure.

Moreover, unless a feature is described as requiring another feature in combination therewith, any feature herein may be combined with any other feature of a same or different embodiment disclosed herein. Furthermore, various well-known aspects of illustrative systems, methods, apparatus, and the like are not described herein in particular detail in order to avoid obscuring aspects of the example embodiments. Such aspects are, however, also contemplated herein.

All references cited in this application are hereby incorporated in their entireties by reference to the extent that they are not inconsistent with the disclosure in this application. It will be apparent to one of ordinary skill in the art that methods, devices, device elements, materials, procedures, and techniques other than those specifically described herein can be applied to the practice of the invention as broadly disclosed herein without resort to undue experimentation. All art-known functional equivalents of methods, devices, device elements, materials, procedures, and techniques specifically described herein are intended to be encompassed by this invention.

When a group of materials, compositions, components, or compounds is disclosed herein, it is understood that all individual members of those groups and all subgroups thereof are disclosed separately. When a Markush group or other grouping is used herein, all individual members of the group and all combinations and sub-combinations possible of the group are intended to be individually included in the disclosure. Every formulation or combination of components described or exemplified herein can be used to practice the invention, unless otherwise stated. Whenever a range is given in the specification, for example, a temperature range, a time range, or a composition range, all intermediate ranges and subranges, as well as all individual values included in the ranges given are intended to be included in the disclosure. All changes which come within the meaning and range of equivalency of the items are to be embraced within their scope.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

What is claimed is:

1. A method of normalizing fluorescence measurements for a multi-channel fluorometer, the method comprising:
receiving a calibrant in a plurality of receptacles of the multi-channel fluorometer;
exciting the plurality of receptacles with one or more light sources;
measuring a fluorescence level of each calibrant in the plurality of receptacles;
using the fluorescence level of the calibrant in a first receptacle of the plurality of receptacles to generate a correction factor for the first receptacle;
creating correction factors for each of the other of the plurality of receptacles using the fluorescence level from the first receptacle;
storing the correction factors; and
applying the correction factors to subsequent measurements of fluorescence levels in each respective receptacle of the plurality of receptacles.

2. The method of claim 1 wherein creating correction factors comprises dividing the fluorescence level of the first receptacle by the fluorescence level of each respective one of the plurality of receptacles.

3. The method of claim 1 further comprising generating a standard curve based at least in part on the correction factors.

4. The method of claim 3 wherein generating the standard curve comprises applying the correction factor obtained for each of the plurality of receptacles to subsequent measurements of fluorescence levels of known concentrations of standard solutions in each respective receptacle of the plurality of receptacles.

5. The method of claim 3 wherein generating the standard curve comprises using two or more replicates of each concentration of standard solution.

6. The method of claim 4, wherein the standard solution comprises a biomolecule.

7. The method of claim 6, wherein the biomolecule is chosen from a protein, a peptide, an amino acid, an enzyme, a toxin, a lectin, a lipopolysaccharide, a nanoparticle, a virus, an extracellular vesicle, a nucleic acid, a polynucleotide, an oligonucleotide, a single-stranded DNA, a double-stranded DNA, an RNA or a microRNA.

8. The method of claim 1 further comprising: obtaining subsequent measurements of fluorescence levels of a sample containing an analyte of interest in one or more receptacle of the plurality of receptacles and applying the correction factor obtained for the one or more receptacle of the plurality of receptacles.

9. The method of claim 8, wherein the analyte of interest is a biomolecule.

10. The method of claim 9, wherein the biomolecule is chosen from a protein, a peptide, an amino acid, an enzyme, a toxin, a lectin, a lipopolysaccharide, a nanoparticle, a virus, an extracellular vesicle, a nucleic acid, a polynucleotide, an oligonucleotide, a single-stranded DNA, a double-stranded DNA, an RNA or a microRNA.

11. The method of claim 1, wherein the calibrant is chosen based on one or more types of light comprising the one or more light sources.

12. The method of claim 11, wherein the calibrant is chosen so that it will fluoresce when excited by the one or more types of light.

13. The method of claim 1, wherein the calibrant comprises a fluorescent dye, fluorophore, quantum dot, fluorescent nanoparticle or fluorescent gelling polymer.

14. A multi-channel fluorometer, comprising:
one or more receptacles configured to receive a test solution therein;
one or more light sources configured to provide excitation to the one or more receptacles;
one or more photodetectors, each photodetector configured to receive emission light from one of the excited receptacles and to measure a fluorescence level of the emission light, and
a non-transitory computer-readable storage medium coupled to the one or more receptacles, light sources and photodetectors, having stored thereon a computer program which, when executed on at least one processor, causes the at least one processor to carry out a method comprising the steps of:
receiving an indication of a calibrant being placed in the one or more receptacles of the multi-channel fluorometer;
exciting the calibrant in the one or more receptacles with the one or more light sources;
measuring the fluorescence level of each calibrant in the one or more receptacles;
using the fluorescence level of the calibrant in a first receptacle of the one or more receptacles to generate a correction factor for the first receptacle;
creating correction factors for each of the other of the one or more receptacles using the fluorescence level from the first receptacle;
storing the correction factors; and
applying the correction factors to subsequent measurements of fluorescence levels in each respective receptacle of the one or more receptacles.

15. The multi-channel fluorometer of claim 14 wherein creating correction factors comprises dividing the fluorescence level of the first receptacle by the fluorescence level each of the other of the one or more receptacles.

16. The multi-channel fluorometer of claim 14 wherein the one or more receptacles comprises eight receptacles.

17. The multi-channel fluorometer of claim 14 wherein the method further comprises storing the first and additional conversion factors until one of the one or more light sources is replaced.

18. The multi-channel fluorometer of claim 14 wherein the method further comprises storing the first and additional conversion factors until the multi-channel fluorometer is powered off.

19. The multi-channel fluorometer of claim 14 wherein the method further comprises storing the first and additional conversion factors until a predetermined length of operation time has been reached for the multi-channel fluorometer.

20. The multi-channel fluorometer of claim 19 wherein the predetermined length of operation time is 10,000 hours.

21. The multi-channel fluorometer of claim 14 wherein the method further comprises displaying on a user interface the measurements of fluorescence levels.

22. The multi-channel fluorometer of claim 14 wherein the method further comprises displaying a graph, on a user interface, of the measurements of fluorescence levels.

23. The multi-channel fluorometer of claim 14 wherein a subsequent measurement comprises a measurement of one or more replicas of a known concentration of a standard solution.

24. The multi-channel fluorometer of claim 22, wherein the standard solution comprises a biomolecule.

25. The multi-channel fluorometer of claim 23, wherein the biomolecule is chosen from a protein, a peptide, an amino acid, an enzyme, a toxin, a lectin, a lipopolysaccharide, a nanoparticle, a virus, an extracellular vesicle, a nucleic acid, a polynucleotide, an oligonucleotide, a single-stranded DNA, a double-stranded DNA, an RNA or a microRNA.

26. The multi-channel fluorometer of claim 14 wherein a subsequent measurement comprises a measurement of a sample comprising an analyte of interest.

27. The multi-channel fluorometer of claim 26 wherein the analyte of interest comprises a biomolecule.

28. The multi-channel fluorometer of claim 27 wherein the biomolecule is chosen from a protein, a peptide, an amino acid, an enzyme, a toxin, a lectin, a lipopolysaccharide, a nanoparticle, a virus, an extracellular vesicle, a nucleic acid, a polynucleotide, an oligonucleotide, a single-stranded DNA, a double-stranded DNA, an RNA or a microRNA.

29. A method of normalizing fluorescence measurements, comprising:
receiving a plurality of fluorescence measurements for a plurality of receptacles in a fluorometer;
using the fluorescence measurement of a first receptacle of the plurality of receptacles to generate a correction factor for the first receptacle;
creating correction factors for each of the other of the plurality of receptacles using the fluorescence measurement from the first receptacle;
storing the correction factors; and
applying the correction factors to a subsequent plurality of fluorescence measurements in each respective receptacle of the plurality of receptacles.

30. The method of claim 29 wherein creating correction factors comprises dividing the fluorescence measurement of the first receptacle by the fluorescence measurement of each respective one of the plurality of receptacles.

31. The method of claim 30 further comprising calculating standard curves for the subsequent plurality of fluorescence measurements.

32. The method of claim 30 further comprising sending the corrected subsequent plurality of fluorescence measurements to a graphical interface.

33. A method of testing fluorescence according to a plurality of standards in a single test, the method comprising:
receiving a first plurality of fluorescence measurements for a plurality of receptacles in a multi-channel fluorometer, each containing a calibrant;
using the fluorescence measurement of a first receptacle of the plurality of receptacles to generate a correction factor for the first receptacle;
creating correction factors for each of the other of the plurality of receptacles using the fluorescence measurement from the first receptacle;
storing the correction factors;
receiving a second plurality of fluorescence measurements for the plurality of receptacles, wherein a first one or more receptacles of the plurality of receptacles contain concentrations of a first standard and a second one or more receptacles of the plurality of receptacles contain concentrations of a second standard; and
applying the correction factors to the second plurality of fluorescence measurements.

34. The method of claim 33, wherein the second plurality of fluorescence measurements further comprises a third one or more receptacles of the plurality of receptacles containing concentrations of a third standard and a fourth one or more receptacles of the plurality of receptacles containing concentrations of a fourth standard.
